(19) United States
(12) Patent Application Publication
(10) Pub. No.: US 2021/0248621 A1
(43) Pub. Date: Aug. 12, 2021

(54) METHODS AND SYSTEMS FOR TRACKING THE TRANSFER OF A CANNABIS ARTICLE

(71) Applicant: Margaret D'Elia, Jeffersonville, VT (US)

(72) Inventor: Margaret D'Elia, Jeffersonville, VT (US)

(21) Appl. No.: 17/156,895

(22) Filed: Jan. 25, 2021

Related U.S. Application Data
(60) Provisional application No. 62/965,412, filed on Jan. 24, 2020.

(51) Int. Cl. G06Q 30/00; H04L 29/06

(52) U.S. Cl. CPC G06Q 30/018; G06N 20/00; H04L 63/08

(57) ABSTRACT

A system for tracking the transfer of a cannabis article. The system includes a computing device that includes a repository module configured to receive at least a user datum from a remote device that includes at least a first user cannabis article. The computing device includes a repository engine operating on the repository module configured to create at least an unspecified datum as a function of at least a user datum, generate a digitally signed assertion, and insert the digitally signed assertion in a temporally sequential listing. The computing device includes a transmission module configured to receive a request for a user datum record, authenticate the request for the user datum record, retrieve a digitally signed assertion containing at least an unspecified datum, and transmit the digitally signed assertion containing an unspecified datum to a remote device.

### CROSS-REFERENCE TO RELATED APPLICATIONS

This application claims the benefit of priority of U.S. Provisional Patent Application Ser. No. 62/965,412, filed on Jan. 24, 2020, and titled “METHODS AND SYSTEMS FOR TRACKING THE TRANSFER OF A CANNABIS ARTICLE,” which is incorporated by reference herein in its entirety.

### FIELD OF THE INVENTION

The present invention generally relates to the field of computer security and secure transactions. In particular, the present invention is directed to methods and systems for tracking transfer of a cannabis article.

### BACKGROUND

Currently cannabis articles are not readily or easily tracked, resulting in both overuse and underuse. Further cannabis article use is shared in an insecure manner with other participants such as insurance companies and research institutions, which can lead to inferior health outcomes.

### SUMMARY OF THE DISCLOSURE

In an aspect, a system for tracking the transfer of a cannabis article, includes a computing device the computing device further comprising a repository module, the repository module designed and configured to receive at least a user datum from a remote device, wherein the at least a user datum includes at least a first user cannabis article, a repository engine operating on the repository module the repository engine designed and configured to receive the at least a user datum from the repository module, create at least an unspecified datum as a function of the at least a user datum, generate a digitally signed assertion containing the at least an unspecified datum, and insert the digitally signed assertion in a temporally sequential listing, and a transmission module the transmission module designed and configured to receive a request from a remote device for a user datum record, authenticate the request from the remote device, retrieve the digitally signed assertion containing the at least an unspecified datum from the repository engine, and transmit the digitally signed assertion containing the at least an unspecified datum to the remote device.

In another aspect, a method for tracking the transfer of a cannabis article includes receiving at a computing device at least a user datum from a remote device, wherein the at least a user datum includes at least a first user cannabis article, creating by the computing device at least an unspecified datum as a function of the at least a user datum, generating by the computing device a digitally signed assertion containing the at least an unspecified datum, inserting by the computing device the digitally signed assertion in a temporally sequential listing, receiving by the computing device a request from a remote device for a user datum record, authenticating by the computing device the request from the remote device, retrieving by the computing device the digitally signed assertion containing the at least an unspecified datum, and transmitting by the computing device the digitally signed assertion containing the at least an unspecified datum to the remote device.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for tracking the transfer of cannabis articles;

FIG. 2 is a block diagram illustrating an exemplary embodiment of a temporally sequential listing of digitally signed assertion;

FIG. 3 is a diagrammatic representation of a system for tracking the transfer of cannabis articles;

FIG. 4 is a block diagram of an exemplary embodiment of a machine-learning module;

FIG. 5 is a process flow diagram illustrating an exemplary embodiment of a method of tracking the transfer of cannabis articles; and

FIG. 6 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for tracking the transfer of a cannabis article. In an embodiment, this disclosure includes receiving at least a user datum from a remote device, wherein the at least a user datum includes at least a first user cannabis article. Aspects of the present disclosure include creating at least an unspecified datum as a function of the at least a user datum. Aspects of the present disclosure can also be used for generating a digitally signed assertion containing the at least an unspecified datum. This is so, at least in part, because the digitally signed assertion is inserted in a temporally sequential listing. Aspects of the present disclosure can be used for receiving a request from a remote device for a user datum record. Aspects of the present disclosure can also be used for authenticating by the computing device the request from the remote device. This is so, at least in part, because the invention utilizes a cryptographic function. Aspects of the present disclosure allow for transmitting the digitally signed assertion containing the at least an unspecified datum to the remote device. Exemplary embodiments illustrating aspects of the present disclosure are described below in the context of several specific examples.

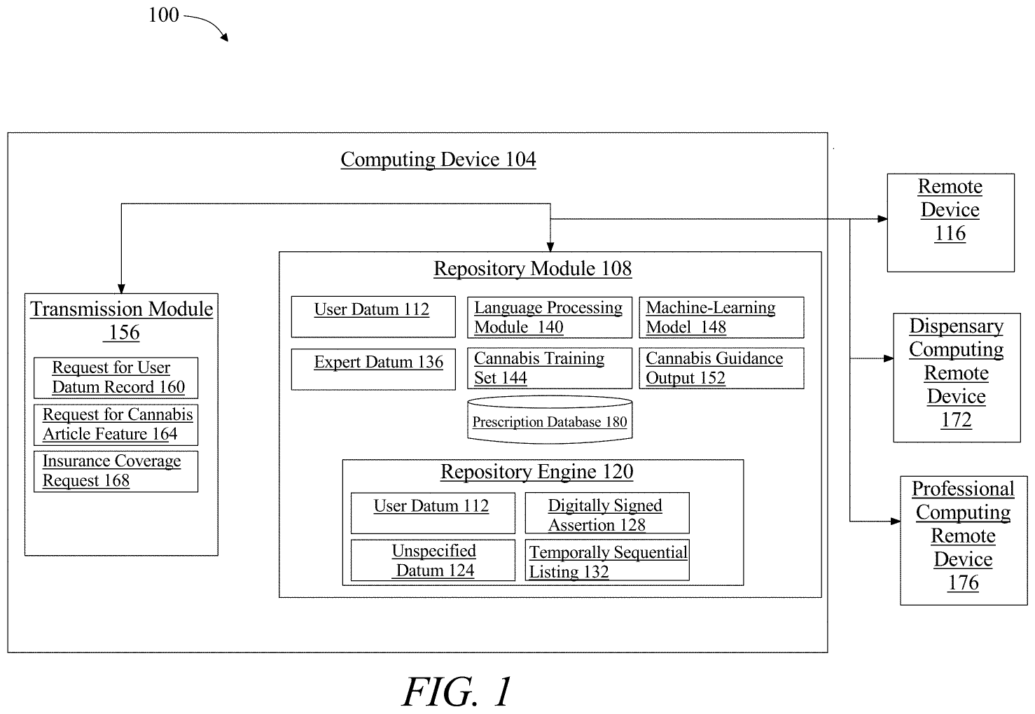

Referring now to FIG. 1, an exemplary embodiment of a system 100 for tracking the transfer of a cannabis article is illustrated. System 100 includes a computing device 104. Computing device 104 may include any computing device 104 as described in this disclosure, including without limitation a microcontroller, microprocessor, digital signal processor (DSP) and/or system on a chip (SoC) as described in this disclosure. Computing device 104 may include, be included in, and/or communicate with a mobile device such as a mobile telephone or smartphone. Computing device 104 may include a single computing device 104 operating independently or may include two or more computing device 104 operating in concert, in parallel, sequentially or the like; two or more computing devices 104 may be included together in a single computing device 104 or in two or more computing devices 104. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices 104, and any combinations thereof. A network may employ a wired and/or a wireless mode of communication. In general, any network topology may be used. Information (e.g., data, software etc.) may be communicated to and/or from a computer and/or a computing device 104. Computing device 104 may include but is not limited to, for example, a computing device 104 or cluster of computing devices 104 in a first location and a second computing device 104 or cluster of computing devices 104 in a second location. Computing device 104 may include one or more computing devices 104 dedicated to data storage, security, distribution of traffic for load balancing, and the like. Computing device 104 may distribute one or more computing tasks as described below across a plurality of computing devices 104 of computing device 104, which may operate in parallel, in series, redundantly, or in any other manner used for distribution of tasks or memory between computing devices 104. Computing device 104 may be implemented using a “shared nothing” architecture in which data is cached at the worker; in an embodiment, this may enable scalability of system 100 and/or computing device 104.

Still referring to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, system 100 includes a repository module 108. Repository module 108 may be configured as any hardware and/or software module. Repository module 108 is designed and configured to receive a user datum 112 from a remote device 116. A “user datum,” as used in this disclosure, is any information relating to a user. A user datum 112 may include personal identifying information such as a user's legal name, birth date, and demographic information such as a user's educational history, marital status, address, occupation, known allergies, emergency contact and the like. A user datum 112 may include information relating to a user's purchase of cannabis products. A “cannabis product” as used in this disclosure is any product derived from a cannabis plant. Cannabis plants may include for example Cannabis sativa, Cannabis Indica, and/or Cannabis Ruderalis. Cannabis products may include one or more psychoactive chemicals including marijuana, hashish, kief, and hash oil. Cannabis products may include one or more active ingredients including delta-9 tetrahydro-cannabinol (THC), delta-8 tetrahydro-cannabinol (d8-THC), hemp, cannabinol, tetrahydrocannabivarin, cannabigerol, cannabichromene, cannabigerivarin, tetrahydrocannabivarin, cannabidivarin, cannabichromevarin, and/or cannabidiol. Cannabis products may be available in one or more dosage forms and/or via one or more delivery mechanisms including cigarettes, pipes, joints, water pipes such as bongs, hookahs, or the like, dried resin, edibles such as gummies, cookies, brownies, capsules, tablets, creams, salves, balms, vapes, tincture, infusions, oil, teas, lotions, liposomes, powders, nanoliposomes, nanoparticles, salts, suspensions, and the like. Cannabis products may be utilized for medical and/or recreational use. Cannabis products may be available for purchase with or without a prescription from a medical professional with prescriptive authority such as a doctor, nurse practitioner, psychiatrist, physician assistant and the like. A user datum 112 may include information relating to a user's purchase of cannabis such as a name of a cannabis product, a medical condition that a user is taking the cannabis product for, information relating to the health history of a user such as previous cannabis products that were previously utilized by the user, and the like. User datum 112 may include information relating to a user's purchase of particular flavors of cannabis products. For example, and without limitation, a user datum may include a user preference and/or consistent purchase of cannabis products that are sweet in flavor. A user datum 112 includes at least a first user cannabis article. A “first user cannabis article,” as used in this disclosure, includes data describing any cannabis product purchased by a user. For instance and without limitation, a first user cannabis article may describe an edible gummy bear containing cannabis that a user purchased. A cannabis product may be purchased in person at any location that sells cannabis products such as a dispensary, store, and/or pharmacy. A cannabis product may be purchased online at any digital location that sells cannabis products such as an online retail dispensary or an online retail pharmacy.

With continued reference to FIG. 1, system 100 includes a remote device 116, which may include any device or devices suitable for use as computing device 104. Remote device 116 may include without limitation, a display in communication with computing device 104, where a display may include any display as described herein. Remote device 116 may include an additional computing device, such as a mobile device, laptop, desktop, computer and the like. Remote device 116 may transmit and/or receive one or more inputs from computing device 104 utilizing any network methodology as described herein. Remote device 116 may be operated by a user which may include any human subject.

With continued reference to FIG. 1, repository module 108 includes a repository engine 120 operating on the repository module 108. Repository engine 120 may be implemented as any hardware and/or software module. Repository engine 120 is configured to receive a user datum 112 from repository module 108. Repository engine 120 may receive a user datum 112 utilizing any network methodology as described herein.

With continued reference to FIG. 1, repository engine 120 is configured to create at least an unspecified datum 124 as a function of at least a user datum 112. An “unspecified datum” as used in this disclosure, is any user datum 112 that does not contain personal identifying information (PII). PII includes any data that could be used to identify a particular person. PII may include a person's full name, social security number, driver's license number, bank account number, passport number, email address, mailing address, and the like. Repository engine 120 may create an unspecified datum 124 by removing PII data pertaining to the user from the at least a user datum 112. For instance and without limitation, repository engine 120 may receive a user datum 112 that contains a user's name, address, age, and a description of a cannabis article purchased by the user at a dispensary. Repository engine 120 may create an unspecified datum 124 by removing PII from the user datum 112 including the user's name and address. In an embodiment, repository engine 120 may not remove a user's age, wherein a user's birthday is removed such that the unspecified datum 124 describes the user as being twenty-seven years old. In an embodiment, repository engine 120 may remove a user's date of birth such as if the user's date of birth is specified as Feb. 26, 1977. In an embodiment, repository engine 120 may remove the year that a user is born but may keep the user's month and date of birth so that a user's birthday that is Feb. 26, 1977 as in the above example, may be modified so that only the year of the user's birthday is removed, and February 26 is retained in an unspecified datum 124.

With continued reference to FIG. 1, repository engine 120 generates a digitally signed assertion 128 containing an unspecified datum and a cannabis guidance article, wherein a cannabis guidance article is any recommendation for any cannabis product, as described below in detail. A “digitally signed assertion,” as used in this disclosure, is a collection of textual data signed using a secure proof as described in further detail below. A digital signature may include, as a non-limiting example, an encrypted mathematical representation of a file or other set of data such as without limitation a cryptographic hash and/or checksum, signed using, for instance, the private key of a public key cryptographic system; in such an example, signature may be verified by decrypting the encrypted mathematical representation using the corresponding public key and comparing the decrypted representation to a purported match that was not encrypted; if the signature protocol is well-designed and implemented correctly, this means the ability to create the digital signature is equivalent to possession of the private decryption key. Likewise, if mathematical representation of file is well-designed and implemented correctly, any alteration of the file will result in a mismatch with the digital signature; the mathematical representation may be produced using an alteration-sensitive, reliably reproducible algorithm, such as a hashing algorithm as described in further detail below. A mathematical representation to which the signature may be compared may be included with signature, for verification purposes; in other embodiments, the algorithm used to produce the mathematical representation is publicly available, permitting the easy reproduction of the mathematical representation corresponding to any file. A digital signature includes a secure proof performed on an element of data, referred to as a “message”; secure proof may include any secure proof as described in this disclosure. Message may include without limitation an encrypted mathematical representation of a file or other set of data. File or set of data may confer credentials, which may demonstrate, without limitation, any result of any authentication or authorization process performed by a signing device. A secure proof may include for example, zero-knowledge proofs.

With continued reference to FIG. 1, in some embodiments, persons, devices, or transactions may be authenticated using digital certificates. In one embodiment, a digital certificate is a file that conveys information and links the conveyed information to a “certificate authority” that is the issuer of a public key in a public key cryptographic system. Certificate authority in some embodiments contains data conveying the certificate authority's authorization for the recipient to perform a task. The authorization may be the authorization to access a given datum. The authorization may be the authorization to access a given process. In some embodiments, the certificate may identify the certificate authority. The digital certificate may include a digital signature.

With continued reference to FIG. 1, in some embodiments, a third party such as a certificate authority (CA) is available to verify that the possessor of the private key is a particular entity; thus, if the certificate authority may be trusted, and the private key has not been stolen, the ability of an entity to produce a digital signature confirms the identity of the entity and links the file to the entity in a verifiable way. Digital signature may be incorporated in a digital certificate, which is a document authenticating the entity possessing the private key by authority of the issuing certificate authority and signed with a digital signature created with that private key and a mathematical representation of the remainder of the certificate. In other embodiments, digital signature is verified by comparing the digital signature to one known to have been created by the entity that purportedly signed the digital signature; for instance, if the public key that decrypts the known signature also decrypts the digital signature, the digital signature may be considered verified. Digital signature may also be used to verify that the file has not been altered since the formation of the digital signature. Although digital signatures have been introduced here as performed using public key cryptographic systems, digital signatures may alternatively or additionally be performed using any zero-knowledge (ZK) proof; for instance, a proof may be recorded in conjunction with a datum, and a verification may be performed by any party seeking to evaluate the proof.

With continued reference to FIG. 1, in some embodiments, systems and methods described herein produce cryptographic hashes, also referred to by the equivalent shorthand term “hashes.” A cryptographic hash, as used herein, is a mathematical representation of a lot of data, such as files or blocks in a block chain as described in further detail below; the mathematical representation is produced by a lossy “one-way” algorithm known as a “hashing algorithm.” Hashing algorithm may be a repeatable process; that is, identical lots of data may produce identical hashes each time they are subjected to a particular hashing algorithm. Because hashing algorithm is lossy, it may be impossible to reconstruct a lot of data from a hash produced from the lot of data using the hashing algorithm. In the case of some hashing algorithms, reconstructing the full lot of data from the corresponding hash using a partial set of data from the full lot of data may be possible only by repeatedly guessing at the remaining data and repeating the hashing algorithm; it is thus computationally difficult if not infeasible for a single computer to produce the lot of data, as the statistical likelihood of correctly guessing the missing data may be extremely low. However, the statistical likelihood of a computer of a set of computers simultaneously attempting to guess the missing data within a useful timeframe may be higher, permitting mining protocols as described in further detail below.

With continued reference to FIG. 1, in an embodiment, hashing algorithm may demonstrate an “avalanche effect,” whereby even extremely small changes to lot of data produce drastically different hashes. This may thwart attempts to avoid the computational work necessary to recreate a hash by simply inserting a fraudulent datum in data lot, enabling the use of hashing algorithms for “tamper-proofing” data such as data contained in an immutable ledger as described in further detail below. This avalanche or “cascade” effect may be evinced by various hashing processes; persons skilled in the art, upon reading the entirety of this disclosure, will be aware of various suitable hashing algorithms for purposes described herein. Verification of a hash corresponding to a lot of data may be performed by running the lot of data through a hashing algorithm used to produce the hash. Such verification may be computationally expensive, albeit feasible, potentially adding up to significant processing delays where repeated hashing, or hashing of large quantities of data, is required, for instance as described in further detail below. Examples of hashing programs include, without limitation, Winternitz hashing algorithms, various generations of Secure Hash Algorithm (including “SHA-1,” “SHA-2,” and “SHA-3”), “Message Digest” family hashes such as “MD4,” “MD5,” “MD6,” and “RIPEMD,” Keccak, “BLAKE” hashes and progeny (e.g., “BLAKE2,” “BLAKE-256,” “BLAKE-512,” and the like), Message Authentication Code (“MAC”)-family hash functions such as PMAC, OMAC, VMAC, HMAC, and UMAC, Polyl305-AES, Elliptic Curve Only Hash (“ECOH”) and similar hash functions, Fast-Syndrome-based (FSB) hash functions, GOST hash functions, the Grøstl hash function, the HAS-160 hash function, the JH hash function, the RadioGatún hash function, the Skein hash function, the Streebog hash function, the SWIFFT hash function, the Tiger hash function, the Whirlpool hash function, or any hash function that satisfies, at the time of implementation, the requirements that a cryptographic hash be deterministic, infeasible to reverse-hash, infeasible to find collisions, and have the property that small changes to an original message to be hashed will change the resulting hash so extensively that the original hash and the new hash appear uncorrelated to each other. A degree of security of a hash function in practice may depend both on the hash function itself and on characteristics of the message and/or digest used in the hash function. For example, where a message is random, for a hash function that fulfills collision-resistance requirements, a brute-force or “birthday attack” may to detect collision may be on the order of O(2^{n/2}) for n output bits; thus, it may take on the order of 2²⁵⁶ operations to locate a collision in a 512 bit output “Dictionary” attacks on hashes likely to have been generated from a non-random original text can have a lower computational complexity, because the space of entries they are guessing is far smaller than the space containing all random permutations of bits. However, the space of possible messages may be augmented by increasing the length or potential length of a possible message, or by implementing a protocol whereby one or more randomly selected strings or sets of data are added to the message, rendering a dictionary attack significantly less effective.

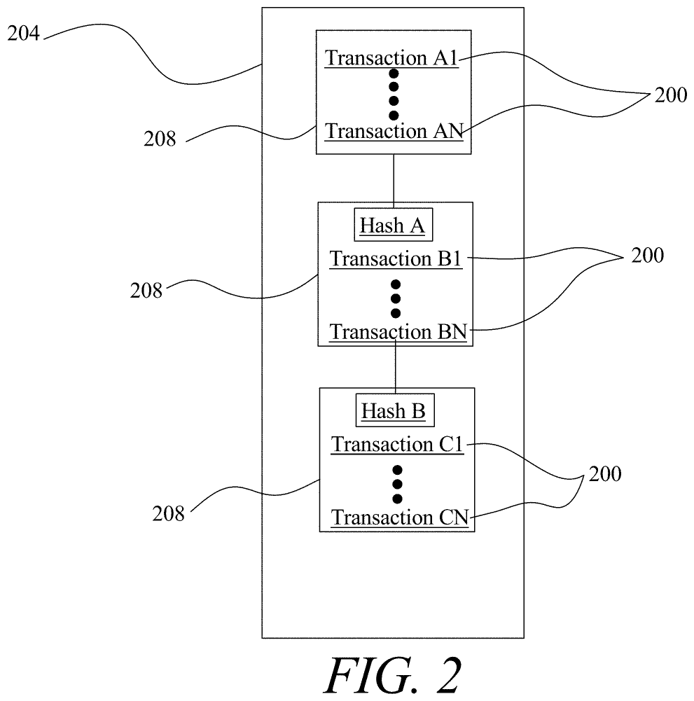

With continued reference to FIG. 1, repository engine 120 is configured to insert a digitally signed assertion 128 into a temporally sequential listing 132. As used in this disclosure a “temporally sequential listing” is a record of a set of data generated by elements or computing devices of system 100. Temporally sequential listing 132 may record data such that it is in an inalterable format, which permits authentication of such entry and may serve as a form of memory storage as described in further detail below. Temporally sequential listing 132 may be accessible at any of various security settings; for instance, and without limitation, the temporally sequential listing 132 may be readable and modifiable publicly, may be publicly readable but writable only by entities and/or devices having access privileges established by password protection, confidence level, or any device authentication procedure or facilities described herein, or may be readable and/or writable only by entities and/or devices having such access privileges. Access privileges may exist in more than one level, including, without limitation, a first access level or community of permitted entities and/or devices having ability to read, and a second access level or community of permitted entities and/or devices having ability to write; first and second community may be overlapping or non-overlapping. Temporally sequential listing 132 may, for instance be encrypted, and decryption keys may be distributed only to devices authorized to participate in authentication as described herein. In an embodiment, decryption key may be stored by transaction authentication nodes as described in further detail below. Exemplary embodiments of temporally sequential listing 132, which may include embodiments of temporally sequential listing 132, are described in more detail below in FIG. 2.

Continuing to look at FIG. 1 temporally sequential listing 132 may be implemented by a plurality of transaction authentication nodes. In an embodiment, a plurality of transaction authentication nodes implementing temporally sequential listing 132 may allow for multiple asset transfers to occur simultaneously. In an embodiment, an asset transfer at a first transaction authentication node may occur while at the same time a second asset transfer may occur at a second transaction authentication node. In an embodiment, a plurality of transaction authentication nodes provides additional levels of security by having additional verifications of accounts. In an embodiment, having a plurality of transaction authentication nodes implementing temporally sequential listing 132 may also allow for simultaneous updates from an institution and allow an institution to generate an approval for more than one transaction authentication node.

With continued reference to FIG. 1, repository module 108 is configured to receive at least an expert datum 136 from a remote device 116. An “expert datum 136” as used in this disclosure, is any healthcare feedback generated in response to a first user cannabis article. Healthcare feedback may be generated by any healthcare professional who may be involved in the user's care, including for example a medical doctor, nurse, pharmacist, nurse practitioner, physician assistant and the like. Healthcare feedback may include information regarding a course of treatment with a first user cannabis article. Healthcare feedback may include effectiveness of a first user cannabis article in controlling a symptom, medical condition, and/or disease state that a healthcare professional may be treating the user for. Healthcare feedback may include a description of one or more side effects the user may be experiencing from taking a cannabis product such as excessive daytime drowsiness at a particular dose or excessive hunger when a user consumes four edibles at once but not when a user consumes three edibles. Healthcare feedback may include information regarding a course of treatment that the user self-reports, such as if the user has an appointment with a healthcare professional and discloses to the healthcare professional a description of how a particular cannabis product reduced a user's anxiety symptoms or that the user consumed the cannabis product as directed, taking it three times each day. Repository module 108 receives an expert datum 136 from a remote device 116. Remote device 116 includes any of the remote device 116 as described above. In an embodiment, remote device 116 may include a computer terminal located in a health professional's office, such as a computing device containing an electronic health record where a health professional may enter one or more expert datums. In an embodiment, remote device 116 may include a mobile phone operated by a healthcare professional or a tablet operated by a healthcare professional.

With continued reference to FIG. 1, expert datum 136 includes a user identifier. A “user identifier” as used in this disclosure, is any identifier that is unique among identifiers used to distinguish a first user from a second user. A user identifier may include serial numbers that may be assigned incrementally or sequentially. A unique identifier may include random numbers selected from a number space that is larger than the maximum or expected number of objects to be identified. Random numbers may include hash functions and/or be selected based on a random number generator that utilizes a random process. A unique identifier may include a name or code that may be selected by a central registry such as by computing device. A unique identifier may include a name or code. A unique identifier may be generated using one or more methods as described above. A unique identifier may include for example, a national identification number, a digital object identifier, a universally unique identifier, a legal entity identifier and the like. A unique identifier may include a public/private key pair, including any of the public/private key pairs as described above.

With continued reference to FIG. 1, repository module 108 generates an expert guidance datum utilizing an expert datum 136. An “expert guidance datum” as used in this disclosure, is any expert datum 136 that does not contain any identifying information relating to a user and/or expert such as a healthcare professional who may be providing healthcare services to a user and who may generate an expert datum 136. Repository module 108 may generate an expert guidance datum by removing any PII and any identifying information relating to an expert such as a doctor's name, address, national provider identifier (NPI), drug enforcement administration (DEA) number and the like. Repository module 108 locates a temporal sequential listing relating to the user as a function of a user identifier. For example, repository module 108 may utilize a user identifier such as a public/private key pair to locate a temporal sequential listing relating to the user. Repository module 108 generates a digitally signed assertion 128 containing an expert guidance datum and stores the digitally signed assertion 128 containing the expert guidance datum linked to a user. An expert guidance datum may be linked to a user where an expert guidance datum contains deidentified information relating to the user. An expert guidance datum may be linked to a user where a temporally sequential listing 132 is determined to be related to an expert guidance datum such as when an expert guidance datum identifies a user and is linked to the same user contained within a user datum 112. Repository module 108 stores a digitally signed assertion 128 containing an expert guidance datum linked to a user on a temporal sequential listing.

With continued reference to FIG. 1, repository module 108 may include a language processing module 140. Repository module 108 may utilize language processing module 140 to map an expert datum 136 to a user condition. “Mapping” as used in this disclosure, is any process that involves identifying a user condition contained within an expert datum 136. A user condition may include any medical condition, diagnosis, indication, and/or reason why a user may be consuming and/or purchasing a cannabis product. For example, a user condition may include a previous diagnosis of generalized anxiety disorder. In yet another non-limiting example, a user condition may include a reason why a user may purchase a cannabis product such as because a user wishes to utilize a cannabis product for recreational purposes.

With continued reference to FIG. 1, language processing module 140 may be configured to extract one or more words from an expert datum 136 and retrieve a user condition. Language processing module 140 may include any hardware and/or software module. Language processing module 140 may be configured to extract, from one or more inputs, one or more words. One or more words may include, without limitation, strings of one or characters, including without limitation any sequence or sequences of letters, numbers, punctuation, diacritic marks, chemical symbols and formulas, spaces, whitespace, and other symbols, including any symbols usable as textual data as described above. Textual data may be parsed into tokens, which may include a simple word (sequence of letters separated by whitespace) or more generally a sequence of characters as described previously. The term “token,” as used herein, refers to any smaller, individual groupings of text from a larger source of text; tokens may be broken up by word, pair of words, sentence, or other delimitation. These tokens may in turn be parsed in various ways. Textual data may be parsed into words or sequences of words, which may be considered words as well. Textual data may be parsed into “n-grams”, where all sequences of n consecutive characters are considered. Any or all possible sequences of tokens or words may be stored as “chains”, for example for use as a Markov chain or Hidden Markov Model.

With continued reference to FIG. 1, language processing module 140 may operate to produce a language processing model. Language processing model may include a program automatically generated by a computing device 104 and/or language processing module 140 to produce associations between one or more words extracted from at least a document and detect associations, including without limitation mathematical associations, between such words, and/or associations of extracted words with categories of user conditions. Associations between language elements, where language elements include for purposes herein extracted words describing and/or including user conditions, including without limitation, mathematical associations, including without limitation statistical correlations between any language element and any other language element and/or language elements. Statistical correlations and/or mathematical associations may include probabilistic formulas or relationships indicating, for instance, a likelihood that a given extracted word indicates a given category of user conditions, and/or a given relationship of such categories to expert datum 136. As a further example, statistical correlations and/or mathematical associations may include probabilistic formulas or relationships indicating a positive and/or negative association between at least an extracted word and/or a given user condition; positive or negative indication may include an indication that a given expert datum 136 is or is not indicating a user condition. For instance, and without limitation, a negative indication may be determined from a phrase such as “user does not report any symptoms related to generalized anxiety disorder,” whereas a positive indication may be determined from a phrase such as “user reports improved sleep duration,” as an illustrative example; whether a phrase, sentence, word, or other textual element in a document or expert datum 136 constitutes a positive or negative indicator may be determined, in an embodiment, by mathematical associations between detected words, comparisons to phrases and/or words indicating positive and/or negative indicators that are stored in memory on a computing device 104, or the like.

Still referring to FIG. 1, language processing module 140 and/or a computing device 104 may generate a language processing model by any suitable method, including without limitation a natural language processing classification algorithm; language processing model may include a natural language process classification model that enumerates and/or derives statistical relationships between input term and output terms. Algorithm to generate language processing model may include a stochastic gradient descent algorithm, which may include a method that iteratively optimizes an objective function, such as an objective function representing a statistical estimation of relationships between terms, including relationships between input terms and output terms, in the form of a sum of relationships to be estimated. In an alternative or additional approach, sequential tokens may be modeled as chains, serving as the observations in a Hidden Markov Model (HMM). HMMs as used herein are statistical models with inference algorithms that that may be applied to the models. In such models, a hidden state to be estimated may include an association between an extracted word category of a user condition, a given relationship of such categories to prognostic labels, and/or a given category of prognostic labels. There may be a finite number of category of physiological data, a given relationship of such categories to prognostic labels, and/or a given category of prognostic labels to which an extracted word may pertain; an HMM inference algorithm, such as the forward-backward algorithm or the Viterbi algorithm, may be used to estimate the most likely discrete state given a word or sequence of words. Language processing module 140 may combine two or more approaches. For instance, and without limitation, machine-learning program may use a combination of Naive-Bayes (NB), Stochastic Gradient Descent (SGD), and parameter grid-searching classification techniques; the result may include a classification algorithm that returns ranked associations.

Continuing to refer to FIG. 1, generating language processing model may include generating a vector space, which may be a collection of vectors, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each vector in an n-dimensional vector space may be represented by an n-tuple of numerical values. Each unique extracted word and/or language element as described above may be represented by a vector of the vector space. In an embodiment, each unique extracted and/or other language element may be represented by a dimension of vector space; as a non-limiting example, each element of a vector may include a number representing an enumeration of co-occurrences of the word and/or language element represented by the vector with another word and/or language element. Vectors may be normalized, scaled according to relative frequencies of appearance and/or file sizes. In an embodiment associating language elements to one another as described above may include computing a degree of vector similarity between a vector representing each language element and a vector representing another language element; vector similarity may be measured according to any norm for proximity and/or similarity of two vectors, including without limitation cosine similarity, which measures the similarity of two vectors by evaluating the cosine of the angle between the vectors, which can be computed using a dot product of the two vectors divided by the lengths of the two vectors. Degree of similarity may include any other geometric measure of distance between vectors.

With continued reference to FIG. 1, repository module 108 is configured to receive a cannabis training set 144. “Cannabis training set 144,” as used in this disclosure, is training data that includes a plurality of data entries including a user attribute label and a correlated cannabis article label. “Training data,” as used in this disclosure, is data containing correlation that a machine-learning process may use to model relationships between two or more categories of data elements. For instance, and without limitation, training data may include a plurality of data entries, each entry representing a set of data elements that were recorded, received, and/or generated together; data elements may be correlated by shared existence in a given data entry, by proximity in a given data entry, or the like. Multiple data entries in training data may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data according to various correlations; correlations may indicate causative and/or predictive links between categories of data elements, which may be modeled as relationships such as mathematical relationships by machine-learning processes as described in further detail below. Training data may be formatted and/or organized by categories of data elements, for instance by associating data elements with one or more descriptors corresponding to categories of data elements. As a non-limiting example, training data may include data entered in standardized forms by persons or processes, such that entry of a given data element in a given field in a form may be mapped to one or more descriptors of categories. Elements in training data may be linked to descriptors of categories by tags, tokens, or other data elements; for instance, and without limitation, training data may be provided in fixed-length formats, formats linking positions of data to categories such as comma-separated value (CSV) formats and/or self-describing formats such as extensible markup language (XML), enabling processes or devices to detect categories of data.

Alternatively or additionally, and still referring to FIG. 1, training data may include one or more elements that are not categorized; that is, training data may not be formatted or contain descriptors for some elements of data. Machine-learning algorithms and/or other processes may sort training data according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data to be made applicable for two or more distinct machine-learning algorithms as described in further detail below. Training data used by a processor may correlate any input data as described in this disclosure to any output data as described in this disclosure.

With continued reference to FIG. 1, repository module 108 creates a first machine-learning model 148 relating an input that includes a user attribute datum to an output that includes a cannabis article using a cannabis training set 144 and a machine-learning algorithm. A first machine-learning model 148 includes any of the machine-learning model 148 as described herein. A “user attribute datum,” as used in this disclosure, is any data relating to a user's health. Data relating to a user's health may include health history, current and/or prior pre-existing medical conditions, current cannabis treatments a user may be consuming and the like.

With continued reference to FIG. 1, repository module 108 is configured to create a first machine-learning model 148 relating a user history datum and a user condition datum to a cannabis article label using a cannabis training set 144 and a machine-learning algorithm. Repository module 108 may create a first machine-learning model 148 using one or more machine-learning processes. A machine learning process, also referred to as a machine-learning algorithm, is a process that automatedly uses training data and/or a training set as described above to generate an algorithm that will be performed by a computing device 104 and/or module to produce outputs given data provided as inputs; this is in contrast to a non-machine learning software program where the commands to be executed are determined in advance by a user and written in a programming language.

Continuing to refer to FIG. 1, machine-learning algorithms may be implemented using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure,

Still referring to FIG. 1, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

With continued reference to FIG. 1, models may be generated using alternative or additional artificial intelligence methods, including without limitation by creating an artificial neural network, such as a convolutional neural network comprising an input layer of nodes, one or more intermediate layers, and an output layer of nodes. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. This network may be trained using training data.

Still referring to FIG. 1, machine-learning algorithms may include supervised machine-learning algorithms. Supervised machine learning algorithms, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised machine-learning process may include a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data. Persons skilled in the art, upon reviewing the entirety of this disclosure, will be aware of various possible variations of supervised machine learning algorithms that may be used to determine relation between inputs and outputs.

With continued reference to FIG. 1, supervised machine-learning processes may include classification algorithms, defined as processes whereby a computing device 104 derives, from training data, a model for sorting inputs into categories or bins of data. Classification may be performed using, without limitation, linear classifiers such as without limitation logistic regression and/or naive Bayes classifiers, nearest neighbor classifiers including without limitation k-nearest neighbors classifiers, support vector machines, decision trees, boosted trees, random forest classifiers, and/or neural network-based classifiers.

Still referring to FIG. 1, machine learning processes may include unsupervised processes. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like. Unsupervised machine-learning algorithms may include, without limitation, clustering algorithms and/or cluster analysis processes, such as without limitation hierarchical clustering, centroid clustering, distribution clustering, clustering using density models, subspace models, group models, graph-based models, signed graph models, neural models, or the like. Unsupervised learning may be performed by neural networks and/or deep learning protocols as described above.

Continuing to refer to FIG. 1, machine-learning processes as described in this disclosure may be used to generate machine-learning model 148. A machine-learning model 148, as used herein, is a mathematical representation of a relationship between inputs and outputs, as generated using any machine-learning process including without limitation any process as described above and stored in memory; an input is submitted to a machine-learning model 148 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 148 may be generated by creating an artificial neural network, such as a convolutional neural network comprising an input layer of nodes, one or more intermediate layers, and an output layer of nodes. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

With continued reference to FIG. 1, repository module 108 generates a cannabis guidance output 152 using the cannabis training set 144 and the first machine-learning model 148. A “cannabis guidance output,” as used in this disclosure, is any recommendation for any cannabis product. Cannabis guidance output 152 may be generated by retrieval, for instance in response to a query, from a prescription database, wherein a prescription database is a database relating to one or more previous cannabis products that a user has purchased, as described below in detail. Cannabis guidance output 152 may be generated from user unspecified data 124. As a non-limiting example, unspecified datum 124 may include previous purchase information associated with a tincture of a hemp product, wherein cannabis guidance output 152 is generated to include a similar tincture of the hemp product. A cannabis guidance output 152 may include a particular brand of a cannabis product, a particular dosage form such as a tincture or edible, a particular dose, a frequency such as how often a user should consume a cannabis product, and the like. For instance and without limitation, a cannabis guidance output 152 may specify a particular cannabis product as well as how often a user should consume the cannabis product. Repository module 108 transmits a cannabis guidance output 152 to a remote device 116. For example, repository module 108 may transmit a cannabis guidance output 152 to a mobile phone operated by a user. In yet another non-limiting example, repository module 108 may transmit a cannabis guidance output 152 to a computer terminal operated by a health professional.

With continued reference to FIG. 1, system 100 includes a transmission module 156. Transmission module 156 may be implemented as any hardware and/or software module. Transmission module 156 is configured to receive a request from a remote device 160 for a user datum 112 record. A “request for a user datum record 160,” as used in this disclosure, is an inquiry to obtain a user datum 112 record. A user datum 112 record may include a history of one or more user cannabis articles. A request for a user datum 112 may be generated by a user at a remote device 116 such as when a user may seek to know his or her own cannabis article history. A request for a user datum 112 may be generated by a health professional at a remote device 116 such as when a user's doctor may seek to inquire about user's cannabis usage. A request for a user datum 112 may be generated by a dispensary or pharmacy that may be seeking to ensure that a user does not purchase excess quantities of cannabis products. Transmission module 156 authenticates a request for a user datum record 160 from a remote device 116. Authenticating a request may include confirming a user identifier such as by authenticating a cryptographic public/private key pair or checking a hash function that represents a user identifier. Authenticating a request may include confirming with a certificate authority that a requestor for a user datum 112 has permission and/or authority to request such information. Transmission module 156 retrieves a digitally signed assertion 128 containing an unspecified datum 124 from repository engine 120 and transmits the digitally signed assertion 128 containing the unspecified datum 124 to the remote device 116. In an embodiment, transmission module 156 may transmit a user datum 112 containing identifying information pertaining to a user when the transmission module 156 has permission from a user to share the user datum 112 with certain parties. For example, user may grant transmission module 156 permission to share a user datum 112 containing user's name and date of birth and cannabis product purchase information with user's medical doctor but not with a telemarketer or a third-party data aggregator.

With continued reference to FIG. 1, transmission module 156 is configured to receive a request from a remote device 116 for a plurality of cannabis article feature 164. A “cannabis article feature,” as used in this disclosure, is any filter that may be applied to any data stored on the temporal sequential listing. A filter may include a request to retrieve information that may relate to usage of a particular cannabis product, use of cannabis products for a selected disease state, use of cannabis products for a selected condition, use of cannabis products by user demographic information such as height, weight, age, and/or gender, use of cannabis products during a selected time frame and the like. Transmission module filters a plurality of digitally signed assertion 128 located on a temporal sequential listing to locate digitally signed assertion 128 that contain a cannabis article feature 164. For instance and without limitation, transmission module 156 may filter a plurality of digitally signed assertions to locate digitally signed assertion 128 that contain all purchase history for the past six months of cannabis vaping products. Transmission module 156 retrieves located digitally signed assertion 128 that contain a specified cannabis article feature 164 and transmit the located digitally signed assertion 128 to the remote device 116. In an embodiment, located digitally signed assertion 128 transmitted to a remote device 116 may contain unspecified datum 124 wherein they do not contain any identifying information pertaining to a particular user.

With continued reference to FIG. 1, transmission module 156 is configured to receive an insurance coverage request 168 for a cannabis article from a remote device 116. An “insurance coverage request,” as used in this disclosure, is a request for third-party payor coverage of a cannabis article. An insurance coverage request 168 may include a request from a user's medical insurance coverage to cover the cost of a cannabis article. Transmission module authenticates the insurance coverage request 168 such as by authenticating a user identifier contained within an insurance coverage request 168. Transmission module 156 transmits a user datum 112 to a remote device 116 that generate an insurance request. For example, transmission module 156 may transmit a user datum 112 containing a user's cannabis article usage over the past four weeks to a remote device 116 such as a computing device operated by the user's health insurance company.

With continued reference to FIG. 1, system 100 may include a dispensary computing remote device 172. Dispensary computing remote device 172 may include any device suitable for use as remote device 116 as described above. Dispensary computing remote device 172 is designed and configured to generate a user datum 112 containing at least a first user cannabis article; display a cannabis tracking consent agreement; receive a user response containing a positive cannabis tracking consent agreement; prompt a user medical professional identification; receive a medical professional identification; and transmit the user datum 112 containing the at least a first user cannabis article and the medical professional identification to a computing device.

With continued reference to FIG. 1, system 100 may include a professional computing remote device 176. Professional computing remote device 176 may include any device suitable for use as remote device 116 as described above. Professional computing remote device 176 is designed and configured to receive from a computing device a digitally signed assertion 128 containing a first user datum 112; generate a second user datum 112 wherein the second user datum 112 contains a response to the first user datum 112; link the first user datum 112 and the second user datum 112; and transmit the linked first user datum 112 and the second user datum 112 to the computing device.

Still referring to FIG. 1, system 100 may include a prescription database 180. Prescription database 180 may be implemented, without limitation, as a relational database, a key-value retrieval database such as a NOSQL database, or any other format or structure for use as a database that a person skilled in the art would recognize as suitable upon review of the entirety of this disclosure. Prescription database 180 may alternatively or additionally be implemented using a distributed data storage protocol and/or data structure. Distributed data storage protocol, as used herein, may include a computer network that contains information that may be stored on more than one node of the data storage, often in a replicated fashion. Distributed data storage protocol may include a non-relational database that enables a quick access to data over a large number of nodes. As a non-limiting example, distributed data storage protocol may include rich query abilities. As a further non-limiting example, distributed data storage protocol may include a distributed hash table, a distributed file system, a peer-to-peer network, and the like thereof. Prescription database 180 may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure. Prescription database may include a unique identifier that at least identifies an object, and/or entity with near certainty that no duplicate has been or will be created. As used in this disclosure “unique identifier” is an n-bit alpha-numeric number that is used to identify information in system 100. As a non-limiting example, unique identifier may include a universally unique identifier (UUID), globally unique identifier (GUID), nil-UUID, version 1 UUID, version 2 UUID, version 3 UUID, version 4 UUID, and the like thereof. Prescription database 180 may include a patient table. As used in this disclosure a “patient table” is a table containing basic patient information. As a non-limiting example, patient table may include a date, patientID, name, Date of birth, gender, height, weight, marijuana card expiration date, status, patient created by, shareable element, and/or primary key. As a further non-limiting example, patient table may be represented as illustrated in the following table:

| | |
|---|---|
| Date | Dec. 17, 2020 |
| PatientID | X8576 |
| Name | John Young Harbringer |
| Date of Birth | Apr. 3, 1974 |
| Gender | Male |
| Height | 5 ft. 9 inches |
| Weight | 187 lbs. |
| Marijuana Card | Jun. 19, 2022 |
| Expiration Date | |
| Status | Active |
| Patient Created By | Dr. Dan S. Cupsworth |
| Shareable Element | Shareable |

Still referring to FIG. 1, prescription database 180 may include a symptom table. As used in this disclosure a “symptom table” is a table containing all symptoms exhibited by a patient. As a non-limiting example, symptom table may include a date, input name, symptom descriptions, symptomID, and/or patientID. As a further non-limiting example, symptom table may be represented as illustrated in the following table:

| | |
|---|---|
| Date | Jan. 11, 2021 |
| Input Name | Dr. Amy L Stallsworth |
| Symptom | Cough, Shortness of |
| Descriptors | Breath, Sneezing, Fever |
| symptomID | s831, s645, s085, s676 |
| patientID | X9456 |

Still referring to FIG. 1, prescription database 180 may include a condition table. As used in this disclosure a “condition table” is a table containing all medical conditions diagnosed for a patient. As a non-limiting example, condition table may include a date, input name, condition description, conditionID, and/or patientID. As a further non-limiting example, condition table may be represented as illustrated in the following table:

| | |
|---|---|
| Date | Jan. 11, 2021 |
| Input Name | Dr. Amy L Stallsworth |
| Condition | Anemia, High Blood |
| Descriptors | pressure, Glaucoma |
| conditionID | c831, c739, c111 |
| patientID | X6301 |

Still referring to FIG. 1, prescription database 180 may include a dispensary table. As used in this disclosure a “dispensary table” is a table containing dispensary elements. As a non-limiting example, dispensary table may include a dispensaryID, name, and/or address. As a further non-limiting example, dispensary table may be represented as illustrated in the following table:

| | |
|---|---|
| dispensaryID | 900-1003-86743 |
| Name | Cannabis Laboratories |
| Address | 123 Light street, Felicity, |
| | Ohio 45120, USA |

Still referring to FIG. 1, prescription database 180 may include a physician table. As used in this disclosure a “physician table” is a table containing physician elements. As a non-limiting example, dispensary table may include a physicianID, name, and/or address. As a further non-limiting example, dispensary table may be represented as illustrated in the following table:

| | |
|---|---|
| physicianID | 46-73-8100 |
| Name | Medi-Cannabis |
| Address | 750 Snowbird lane, |
| | Douglasville, Georgia, 30134, USA |

Still referring to FIG. 1, prescription database 180 may include a transaction table. As used in this disclosure a “transaction table” is a table containing transaction elements representing cannabis purchases and/or sales. As a non-limiting example, transaction table may include a product name, consumption category, active ingredient, dose, frequency, tracking number, patientID, physyicianID, and/or dispensaryID. As a further non-limiting example, dispensary table may be represented as illustrated in the following table:

| Product Name | Green Cush |
|---|---|
| Consumption | Ingestible |
| Category | |
| Active Ingredient | Cannabidiol |
| Dose | 25 mg |
| Frequency | 4 hours |
| Tracking number | 9001-8367-8267-0947 |
| patientID | X6301 |
| physicianID | 46-73-8100 |
| dispensarylD | 900-1003-86743 |

Referring now to FIG. 2, system 100 may be used to perform one or more processing steps necessary to create, maintain, and/or authenticate a digitally signed assertion 200. In an embodiment, at least a digitally signed assertion 200 is a collection of textual data signed using a secure proof as described in further detail below; secure proof may include, without limitation, a digital signature as described above. Collection of textual data may contain any textual data, including without limitation American Standard Code for Information Interchange (ASCII), Unicode, or similar computer-encoded textual data, any alphanumeric data, punctuation, diacritical mark, or any character or other marking used in any writing system to convey information, in any form, including any plaintext or cyphertext data; in an embodiment, collection of textual data may be encrypted, or may be a hash of other data, such as a root or node of a Merkle tree or hash tree, or a hash of any other information desired to be recorded in some fashion using a digitally signed assertion 200. In an embodiment, collection of textual data states that the owner of a certain transferable item of value represented in the at least a digitally signed assertion 200 register is transferring that item to the owner of an address. At least a digitally signed assertion 200 may be signed by a digital signature created using the private key associated with the owner's public key, as described above. For instance, at least a digitally signed assertion 200 may describe a transfer of virtual currency, such as crypto currency as described below. The virtual currency may be a digital currency. Item of value may be a transfer of trust, for instance represented by a statement vouching for the identity or trustworthiness of the first entity. Item of value may be an interest in a fungible negotiable financial instrument representing ownership in a public or private corporation, a creditor relationship with a governmental body or a corporation, rights to ownership represented by an option, derivative financial instrument, commodity, debt-backed security such as a bond or debenture or other security as described in further detail below. At least a digitally signed assertion 200 may describe the transfer of a physical good; for instance, at least a digitally signed assertion 200 may describe the sale of a product. In some embodiments, a transfer nominally of one item may be used to represent a transfer of another item; for instance, a transfer of virtual currency may be interpreted as representing a transfer of an access right; conversely, where the item nominally transferred is something other than virtual currency, the transfer itself may still be treated as a transfer of virtual currency, having value that depends on many potential factors including the value of the item nominally transferred and the monetary value attendant to having the output of the transfer moved into a particular user's control. The item of value may be associated with the at least a digitally signed assertion 200 by means of an exterior protocol, such as the COLORED COINS created according to protocols developed by The Colored Coins Foundation, the MASTERCOIN protocol developed by the Mastercoin Foundation, or the ETHEREUM platform offered by the Stiftung Ethereum Foundation of Baar, Switzerland, the Thunder protocol developed by Thunder Consensus, or any other protocol.

Still referring to FIG. 2, in an embodiment, an address is a textual datum identifying the recipient of virtual currency or another item of value in at least a digitally signed assertion 200. Address may be linked to a public key, the corresponding private key of which is owned by the recipient of the at least a digitally signed assertion 200. For instance, address may be the public key. Address may be a representation, such as a hash, of the public key. Address may be linked to the public key in memory of a computing device, for instance via a “wallet shortener” protocol. Where address is linked to a public key, a transferee in at least a digitally signed assertion 200 may record a subsequent at least a digitally signed assertion 200 transferring some or all of the value transferred in the first at least a digitally signed assertion 200 to a new address in the same manner. At least a digitally signed assertion 200 may contain textual information that is not a transfer of some item of value in addition to, or as an alternative to, such a transfer. For instance, as described in further detail below, at least a digitally signed assertion 200 may indicate a confidence level associated with a cryptographic evaluator as described in further detail below.

With continued reference to FIG. 2, at least a digitally signed assertion 200 may be included in a temporally sequential listing 204. Temporally sequential listing 204 may include any set of data used to record a series of at least a digitally signed assertion 200 in an inalterable format that permits authentication of such at least a digitally signed assertion 200. In some embodiments, temporally sequential listing 204 records a series of digitally signed assertion 128 of at least a digitally signed assertion 200 in a way that preserves the order in which the at least a digitally signed assertion 200 took place. Temporally sequential listing 132 may be accessible at any of various security settings; for instance, and without limitation, temporally sequential listing 132 may be readable and modifiable publicly, may be publicly readable but writable only by entities and/or devices having access privileges established by password protection, confidence level, or any device authentication procedure or facilities described herein, or may be readable and/or writable only by entities and/or devices having such access privileges. Access privileges may exist in more than one level, including, without limitation, a first access level or community of permitted entities and/or devices having ability to read, and a second access level or community of permitted entities and/or devices having ability to write; first and second community may be overlapping or non-overlapping.

Still referring to FIG. 2, temporally sequential listing 204 may preserve the order in which the at least a digitally signed assertion 200 took place by listing them in chronological order; alternatively or additionally, temporally sequential listing 204 may organize digitally signed assertion 200 into sub-listings 208 such as “blocks” in a blockchain, which may be themselves collected in a temporally sequential order; digitally signed assertion 200 within a sub-listing 208 may or may not be temporally sequential. Temporally sequential listing 204 may preserve the order in which at least a digitally signed assertion 200 took place by listing them in sub-listings 208 and placing the sub-listings 208 in chronological order. The temporally sequential listing 204 may be a distributed, consensus-based ledger, such as those operated according to the protocols promulgated by Ripple Labs, Inc., of San Francisco, Calif., or the Stellar Development Foundation, of San Francisco, Calif., or of Thunder Consensus, or Hyperledger, Hyperledger Sawtooth or the like. In an embodiment, temporally sequential listing 204 may be a secured ledger; in one embodiment, a secured ledger is a ledger having safeguards against alteration by unauthorized parties. Temporally sequential listing 204 may be maintained by a proprietor, such as a system administrator on a server, that controls access to the ledger; for instance, the user account controls may allow contributors to the ledger to add at least a digitally signed assertion 200 to the ledger but may not allow any users to alter at least a digitally signed assertion 200 that have been added to the ledger. In an embodiment, temporally sequential listing 204 may be cryptographically secured; in one embodiment, a ledger is cryptographically secured where each link in the chain contains encrypted or hashed information that makes it practically infeasible to alter the ledger without betraying that alteration has taken place, for instance by requiring that an administrator or other party sign new additions to the chain with a digital signature. Temporally sequential listing 204 may be incorporated in, stored in, or incorporate, any suitable data structure, including without limitation any database, datastore, file structure, distributed hash table, or the like. As used in this disclosure a “distributed hash table” is a distributed system that provides a lookup service for key-pair values stored. As a non-limiting example, temporally sequential listing 204 may be stored in a distributed hash table (DHT) such that minimum work to add and/or remove keys is required to update the map to values associated with user datum. In some embodiments, the timestamp of an entry is cryptographically secured and validated via trusted time, either directly on the chain or indirectly by utilizing a separate chain. In one embodiment the validity of timestamp is provided using a time stamping authority as described in the RFC 3161 standard for trusted timestamps, or in the ANSI ASC x9.95 standard. In another embodiment, trusted time ordering is provided by a group of entities collectively acting as the time stamping authority with a requirement that a threshold number of the group of authorities sign the timestamp.

In some embodiments, and with continued reference to FIG. 2, temporally sequential listing 204, once formed, cannot be altered by any party, no matter what access rights that party possesses. For instance, temporally sequential listing 204 may include a one-way cryptographic function. As used in this disclosure a “one-way cryptographic function” is an algorithm that maps data of an arbitrary size to a bit array of a fixed size, which is practically infeasible to invert. As a non-limiting example one-way cryptographic function may include a hash chain, in which data is added during a successive hashing process to ensure non-repudiation. Temporally sequential listing 204 may include a block chain. In one embodiment, a block chain is temporally sequential listing 204 that records one or more new at least a digitally signed assertion 200 in a data item known as a sub-listing 208 or “block.” An example of a block chain is the BITCOIN block chain used to record BITCOIN transactions and values. Sub-listings 208 may be created in a way that places the sub-listings 208 in chronological order and links each sub-listing 208 to a previous sub-listing 208 in the chronological order, so that any computing device may traverse the sub-listings 208 in reverse chronological order to verify any at least a digitally signed assertion 200 listed in the block chain. Each new sub-listing 208 may be required to contain a cryptographic hash describing the previous sub-listing 208. In some embodiments, the block chain contains a single first sub-listing 208 sometimes known as a “genesis block.”

Still referring to FIG. 2, the creation of a new sub-listing 208 may be computationally expensive; for instance, the creation of a new sub-listing 208 may be designed by a “proof of work” protocol accepted by all participants in forming the temporally sequential listing 204 to take a powerful set of computing devices a certain period of time to produce. Where one sub-listing 208 takes less time for a given set of computing devices to produce the sub-listing 208 protocol may adjust the algorithm to produce the next sub-listing 208 so that it will require more steps; where one sub-listing 208 takes more time for a given set of computing devices to produce the sub-listing 208 protocol may adjust the algorithm to produce the next sub-listing 208 so that it will require fewer steps. As an example, protocol may require a new sub-listing 208 to contain a cryptographic hash describing its contents; the cryptographic hash may be required to satisfy a mathematical condition, achieved by having the sub-listing 208 contain a number, called a nonce, whose value is determined after the fact by the discovery of the hash that satisfies the mathematical condition. Continuing the example, the protocol may be able to adjust the mathematical condition so that the discovery of the hash describing a sub-listing 208 and satisfying the mathematical condition requires more or less steps, depending on the outcome of the previous hashing attempt. Mathematical condition, as an example, might be that the hash contains a certain number of leading zeros and a hashing algorithm that requires more steps to find a hash containing a greater number of leading zeros, and fewer steps to find a hash containing a lesser number of leading zeros. In some embodiments, production of a new sub-listing 208 according to the protocol is known as “mining.” The creation of a new sub-listing 208 may be designed by a “proof of stake” protocol as will be apparent to those skilled in the art upon reviewing the entirety of this disclosure. Alternatively, creation of new sublisting 208 may be performed according to proof of elapsed time or other random beacon-based assignment mechanisms, by proof of authority, or the like.

Continuing to refer to FIG. 2, in some embodiments, protocol also creates an incentive to mine new sub-listings 208. The incentive may be financial; for instance, successfully mining a new sub-listing 208 may result in the person or entity that mines the sub-listing 208 receiving a predetermined amount of currency. The currency may be fiat currency. Currency may be crypto-currency as defined below. Incentive may be redeemed for particular products or services; the incentive may be a gift certificate with a particular business, for instance. Incentive may be sufficiently attractive to cause participants to compete for the incentive by trying to race each other to the creation of sub-listings 208 Each sub-listing 208 created in temporally sequential listing 204 may contain a record or at least a digitally signed assertion 200 describing one or more addresses that receive an incentive, such as virtual currency, as the result of successfully mining the sub-listing 208.

With continued reference to FIG. 2, where two entities simultaneously create new sub-listings 208, temporally sequential listing 204 may develop a fork; protocol may determine which of the two alternate branches in the fork is the valid new portion of the temporally sequential listing 204 by evaluating, after a certain amount of time has passed, which branch is longer. “Length” may be measured according to the number of sub-listings 208 in the branch. Length may be measured according to the total computational cost of producing the branch. Protocol may treat only at least a digitally signed assertion 200 contained the valid branch as valid at least a digitally signed assertion 200. When a branch is found invalid according to this protocol, at least a digitally signed assertion 200 registered in that branch may be recreated in a new sub-listing 208 in the valid branch; the protocol may reject “double spending” at least a digitally signed assertion 200 that transfer the same virtual currency that another at least a digitally signed assertion 200 in the valid branch has already transferred. As a result, in some embodiments the creation of fraudulent at least a digitally signed assertion 200 requires the creation of a longer temporally sequential listing 204 branch by the entity attempting the fraudulent at least a digitally signed assertion 200 than the branch being produced by the rest of the participants; as long as the entity creating the fraudulent at least a digitally signed assertion 200 is likely the only one with the incentive to create the branch containing the fraudulent at least a digitally signed assertion 200, the computational cost of the creation of that branch may be practically infeasible, guaranteeing the validity of all at least a digitally signed assertion 200 in the temporally sequential listing 204.

Still referring to FIG. 2, additional data linked to at least a digitally signed assertion 200 may be incorporated in sub-listings 208 in the temporally sequential listing 204; for instance, data may be incorporated in one or more fields recognized by block chain protocols that permit a person or computer forming a at least a digitally signed assertion 200 to insert additional data in the temporally sequential listing 204. In some embodiments, additional data is incorporated in an unspendable at least a digitally signed assertion 200 field. For instance, the data may be incorporated in an OP_RETURN within the BITCOIN block chain. Additional data may be incorporated in one signature of a multi-signature at least a digitally signed assertion 200. In an embodiment, a multi-signature at least a digitally signed assertion 200 is at least a digitally signed assertion 200 to two or more addresses. In an embodiment, two or more addresses may be hashed together to form a single address, which is signed in digital signature of at least a digitally signed assertion 200. Two or more addresses may be concatenated. Two or more addresses may be combined by a more complicated process, such as the creation of a Merkle tree or the like. One or more addresses incorporated in the multi-signature at least a digitally signed assertion 200 may include typical crypto-currency addresses, such as addresses linked to public keys as described above, while one or more additional addresses in the multi-signature at least a digitally signed assertion 200 may contain additional data related to the at least a digitally signed assertion 200; for instance, the additional data may indicate the purpose of the at least a digitally signed assertion 200, aside from an exchange of virtual currency, such as the item for which the virtual currency was exchanged. Additional information may include network statistics for a given node of network, such as a cryptographic evaluator, e.g. the latencies to nearest neighbors in a network graph, the identities or identifying information of neighboring nodes in the network graph, the trust level and/or mechanisms of trust (e.g. certificates of physical encryption keys, certificates of software encryption keys, (in non-limiting example certificates of software encryption may indicate the firmware version, manufacturer, hardware version and the like), certificates from a trusted third party, certificates from a decentralized anonymous authentication procedure, and other information quantifying the trusted status of the cryptographic evaluator) of neighboring nodes in the network graph, IP addresses, GPS coordinates, and other information informing location of the node and/or neighboring nodes, geographically and/or within the network graph. In some embodiments, additional information may include history and/or statistics of neighboring nodes with which the node has interacted. This additional information may be encoded directly, via a hash, hash tree or other encoding.

Still referring to FIG. 1, digitally signed assertion 200 may be traded as a crypto currency. In one embodiment, a crypto currency is a digital, currency such as Bitcoins, Peercoins, Namecoins, and Litecoins. Crypto-currency may be a clone of another crypto-currency. The crypto-currency may be an “alt-coin.” Crypto-currency may be decentralized, with no particular entity controlling it; the integrity of the crypto-currency may be maintained by adherence by its participants to established protocols for exchange and for production of new currency, which may be enforced by software implementing the crypto-currency. Crypto currency may be centralized, with its protocols enforced or hosted by a particular entity. For instance, crypto currency may be maintained in a centralized ledger, as in the case of the XRP currency of Ripple Labs, Inc., of San Francisco, Calif. In lieu of a centrally controlling authority, such as a national bank, to manage currency values, the number of units of a particular crypto-currency may be limited; the rate at which units of crypto-currency enter the market may be managed by a mutually agreed-upon process, such as creating new units of currency when mathematical puzzles are solved, the degree of difficulty of the puzzles being adjustable to control the rate at which new units enter the market. Mathematical puzzles may be the same as the algorithms used to make productions of sub-listings 208 in a block chain computationally challenging; the incentive for producing sub-listings 208 may include the grant of new crypto currency to the miners. Quantities of crypto currency may be exchanged using at least a digitally signed assertion 200 as described above.

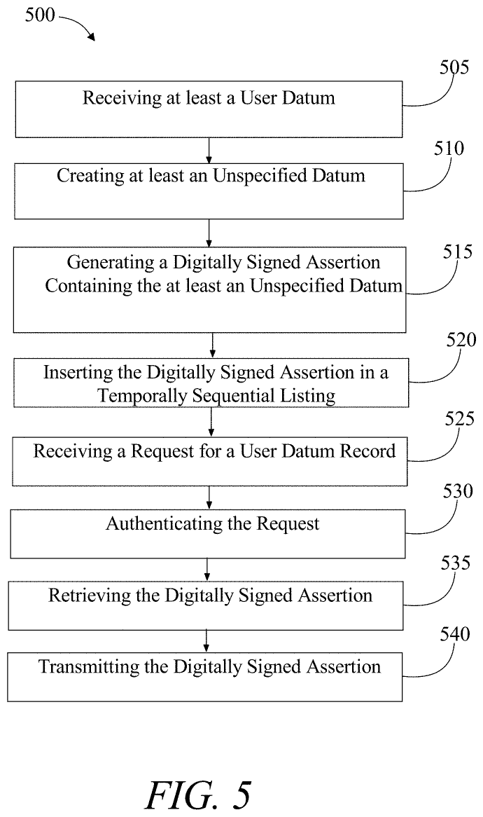

Still referring to FIG. 2, at least a digitally signed assertion 200 may be included data structures or memory elements besides a temporally sequential file, including without limitation any temporary or persistent memory as used in or by any computing device as described below in reference to FIG. 5.

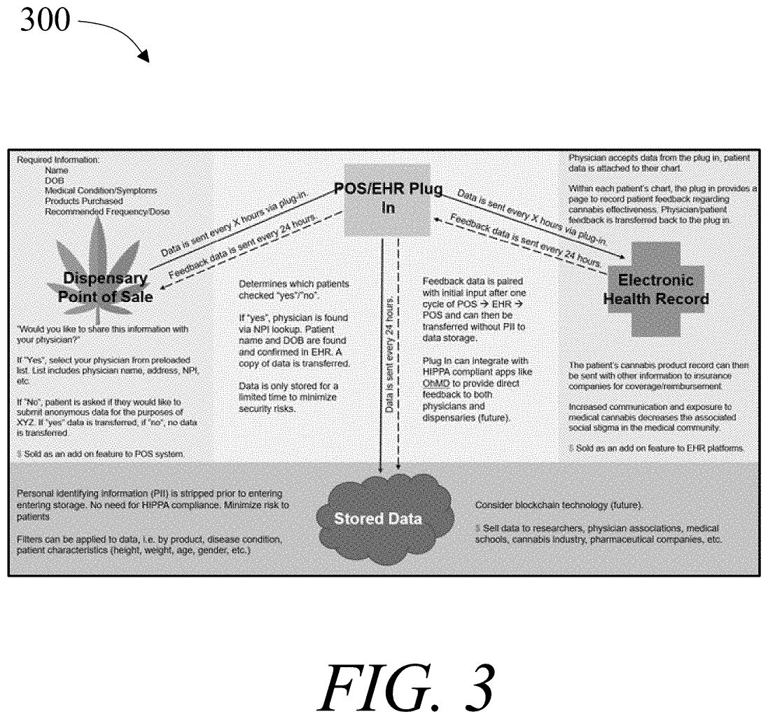

Referring now to FIG. 3, a diagrammatic representation 300 of a system for tracking the transfer of a cannabis article is illustrated. In an embodiment, a user datum 112 may be generated at a dispensary point of sale. User datum 112 may include information such as a user's name, date of birth, medical conditions, symptoms, products purchased, recommended frequency, and recommended dose. Dispensary point of sale may display a cannabis tracking consent agreement that may allow for a user to share information regarding cannabis articles that a user may purchase at a dispensary with a user's health professionals such as use's doctor. Upon receiving a positive confirmation or yes from a user to consent to a cannabis tracking consent agreement, dispensary point of sale may prompt a user medical professional identification where a user can select his or her physician from a preloaded list that may contain a physician name, address, national provider identification (NPI) and the like. If a user does not indicate a positive confirmation to consent to a cannabis tracking consent agreement, a user may then be prompted to determine if the user would like to submit anonymous data. Dispensary point of sale transmits a user datum 112 containing a first user cannabis article and a medical professional identification to a computing device 104 or POS/HER Plug in. Computing device 104 may remove personal identifying information contained within a user datum 112 prior to storage. User datum 112 may be stored as a digitally signed assertion 128 and inserted into a temporal sequential listing. Computing device 104 may be configured to integrate with health insurance portability and accountability act (HIPPA) compliant applications such as OhMD as produced by OhMD of New York, N.Y. Information stored on a temporal sequential listing may be utilized by researchers, physician associations, medical schools, cannabis industry, pharmaceutical companies and the like. Electronic health record may include a separate device which may include any device suitable for use as remote device 116. Electronic health record may receive from computing device 104 a digitally signed assertion 128 containing a first user datum 112. Electronic health record may generate a second user datum 112 wherein the second user datum 112 contains a response to the first user datum 112. A response may include any patient feedback regarding cannabis effectiveness. Electronic health record may link a first user datum 112 and a second user datum 112 and transmit the linked first user datum and the second user datum 112 to computing device 104. User datum 112 may be transmitted to other devices such as to an insurance company device to provide coverage of a cannabis article.

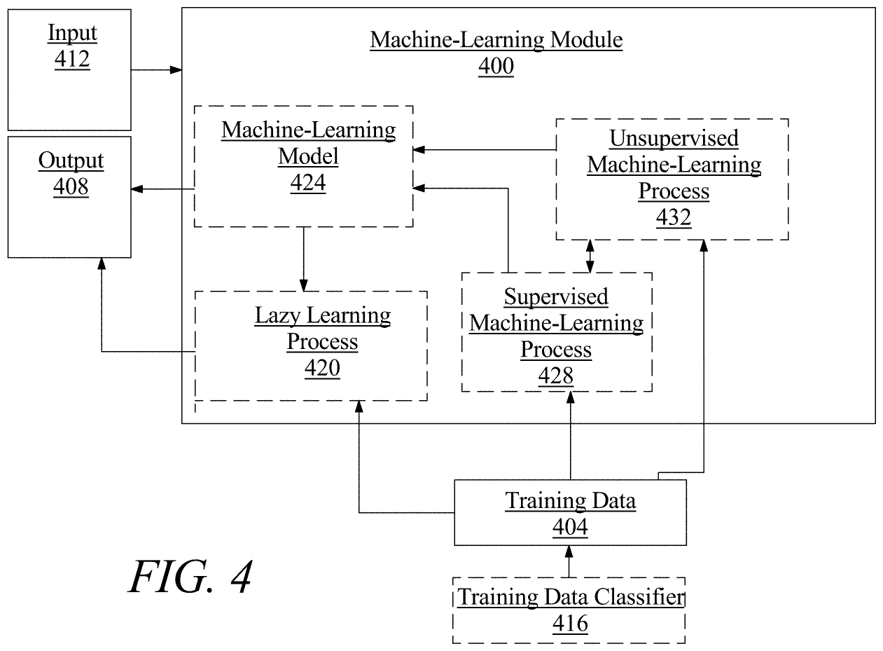

Referring now to FIG. 4, Referring now to FIG. 4, an exemplary embodiment of a machine-learning module 400 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 404 to generate an algorithm that will be performed by a computing device/module to produce outputs 408 given data provided as inputs 412; this is in contrast to a non-machine learning software program where the commands to be executed are determined in advance by a user and written in a programming language.

Still referring to FIG. 4, “training data,” as used herein, is data containing correlations that a machine-learning process may use to model relationships between two or more categories of data elements. For instance, and without limitation, training data 404 may include a plurality of data entries, each entry representing a set of data elements that were recorded, received, and/or generated together; data elements may be correlated by shared existence in a given data entry, by proximity in a given data entry, or the like. Multiple data entries in training data 404 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 404 according to various correlations; correlations may indicate causative and/or predictive links between categories of data elements, which may be modeled as relationships such as mathematical relationships by machine-learning processes as described in further detail below. Training data 404 may be formatted and/or organized by categories of data elements, for instance by associating data elements with one or more descriptors corresponding to categories of data elements. As a non-limiting example, training data 404 may include data entered in standardized forms by persons or processes, such that entry of a given data element in a given field in a form may be mapped to one or more descriptors of categories. Elements in training data 404 may be linked to descriptors of categories by tags, tokens, or other data elements; for instance, and without limitation, training data 404 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 4, training data 404 may include one or more elements that are not categorized; that is, training data 404 may not be formatted or contain descriptors for some elements of data. Machine-learning algorithms and/or other processes may sort training data 404 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 404 to be made applicable for two or more distinct machine-learning algorithms as described in further detail below. Training data 404 used by machine-learning module 400 may correlate any input data as described in this disclosure to any output data as described in this disclosure. As a non-limiting illustrative example a user attribute datum input may result in an output of a cannabis article.

Further referring to FIG. 4, training data may be filtered, sorted, and/or selected using one or more supervised and/or unsupervised machine-learning processes and/or models as described in further detail below; such models may include without limitation a training data classifier 416. Training data classifier 416 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 400 may generate a classifier using a classification algorithm, defined as a processes whereby a computing device and/or any module and/or component operating thereon derives a classifier from training data 404. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 416 may classify elements of training data to sub-categories of user attribute datum such as a name detail, product detail, ailment detail, vigor history detail, and/or a dose detail.

Still referring to FIG. 4, machine-learning module 400 may be configured to perform a lazy-learning process 420 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 404. Heuristic may include selecting some number of highest-ranking associations and/or training data 404 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 4, machine-learning processes as described in this disclosure may be used to generate machine-learning models 424. A “machine-learning model,” as used in this disclosure, is a mathematical and/or algorithmic representation of a relationship between inputs and outputs, as generated using any machine-learning process including without limitation any process as described above and stored in memory; an input is submitted to a machine-learning model 424 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 424 may be generated by creating an artificial neural network, such as a convolutional neural network comprising an input layer of nodes, one or more intermediate layers, and an output layer of nodes. Connections between nodes may be created via the process of “training” the network, in which elements from a training data 404 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 4, machine-learning algorithms may include at least a supervised machine-learning process 428. At least a supervised machine-learning process 428, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include user attribute data as described above as inputs, cannabis articles as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 404. Persons skilled in the art, upon reviewing the entirety of this disclosure, will be aware of various possible variations of at least a supervised machine-learning process 428 that may be used to determine relation between inputs and outputs. Supervised machine-learning processes may include classification algorithms as defined above.

Further referring to FIG. 4, machine learning processes may include at least an unsupervised machine-learning processes 432. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 4, machine-learning module 400 may be designed and configured to create a machine-learning model 424 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 4, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

Referring now to FIG. 5, an exemplary embodiment of a method 500 of tracking the transfer of a cannabis article is illustrated. At step 505 a computing device 104 receives at least a user datum 112 from a remote device 116. Computing device 104 may receive at least a user datum 112 from a remote device 116 utilizing any network methodology as described herein. At least a user datum 112 includes at least a first user cannabis article. A first user cannabis article may include a description of one or more cannabis products purchased by a user. For instance and without limitation, a first user cannabis article may include cannabidiol 25 mg capsules that a user purchased from an online dispensary. In yet another non-limiting example, a first user cannabis article may include a tetrahydrocannabinol (THC) 3% topical salve that a user may purchase from a compounding pharmacy to be applied to user's neck three times daily. At least a user datum 112 may include information pertaining to the user such as a username datum, a user birth datum, a user product datum, a user condition datum, a user health history datum, and a use dose datum.

With continued reference to FIG. 5, at step 510 a computing device 104 creates at least an unspecified datum 124 as a function of a user datum 112. An unspecified datum 124 includes any user datum 112 that does not contain personal identifying information (PII) as described above in more detail. In an embodiment, computing device 104 may create an unspecified datum 124 by removing personal identifying data pertaining to a user from at least a user datum 112. For instance and without limitation, computing device 104 may remove PII such as a user's full legal name and address, or a user's social security number or driver license number. Computing device 104 generates a digitally signed assertion 128 containing a unique identifier for a user. Digitally signed assertion 128 includes any of the digitally signed assertion 128 as described above in more detail in reference to FIG. 2. Unique identifier may include any of the unique identifiers as described above, including for example a series of random numbers or a national identification number or a cryptographic public/private key pair. Computing device 104 inserts a digitally signed assertion 128 containing a unique identifier in a temporally sequential listing 132. Computing device 104 stores user identifying data separate from deidentified user information. In an embodiment, computing device 104 stores user identifying data in repository module 108.

With continued reference to FIG. 5, at step 515 computing device 104 generates a digitally signed assertion 128 containing an unspecified datum 124. This may be performed utilizing any of the methodologies as described above in reference to FIGS. 1-4. Digitally signed assertion 128 is generated to contain a timestamp. In an embodiment, a secure timestamp may be generated when a user datum 112 is received from remote device 116. In an embodiment, a secure timestamp may be generated when computing device 104 generates a digitally signed assertion 128. Computing device 104 generates a digitally signed assertion 128 to contain a secure timestamp.

With continued reference to FIG. 5, computing device 104 may receive an expert datum 136 from a remote device 116. Expert datum 136 includes any healthcare feedback generated in response to a first user cannabis article as described above in more detail in reference to FIG. 1. Expert datum 136 may be generated based on one or more user responses disclosed to a healthcare professional such as user's physician or a nurse. Expert datum 136 may be generated by computing device 104 by mapping utilizing language processing module 140 an expert datum 136 to a user condition. For instance and without limitation, an expert datum 136 that contains a healthcare response to a user's use of cannabidiol (CBD) capsules for irritable bowel syndrome (IBS) may be mapped to IBS utilizing language processing module 140. Such information may be linked and inserted into temporal sequential listing to be utilized later on such as for an observational study or retrospective analysis. Expert datum 136 includes a user identifier, which may include any of the user identifiers as described above. Computing device 104 utilizes an expert datum 136 to generate an expert guidance datum. Expert datum 136 does not contain any identifying information relating to a user and/or expert such as a healthcare professional who may be providing healthcare services to a user and only contains a response generated to a first user cannabis article. In an embodiment, expert datum 136 may contain an identified first user cannabis article. Computing device 104 locates a temporal sequential listing relating to a user as a function of a user identifier contained within an expert datum 136. For instance and without limitation, computing device 104 may identify a temporal sequential listing that contains a user identifier that matches a user identifier contained within an expert datum 136 such as by matching a cryptographic public/private key pair or by authenticating a hash. Computing device 104 generates a digitally signed assertion 128 containing an expert guidance datum and stores the digitally signed assertion 128 containing an expert guidance datum linked to the user in the temporal sequential listing. In an embodiment, a temporal sequential listing that contains a user identifier that matches a user identifier contained within an expert datum 136 may be inserted and stored together into a temporal sequential listing.

With continued reference to FIG. 5, at step 520 computing device 104 inserts a digitally signed assertion into a temporally sequential listing 132. This may be performed utilizing any methodology as described above in reference to FIGS. 1-4.

With continued reference to FIG. 5, at step 525 computing device 104 receives a request from a remote device 116 for a user datum 112 record. A user datum 112 record may include a history of one or more user cannabis articles as described above in more detail in reference to FIG. 1. A request for a user datum record 160 may be generated from a remote device 116, such as when user's doctor may generate a request to evaluate user's cannabis history and use over the past one year.

With continued reference to FIG. 5, at step 530 computing device 104 authenticates a request for a remote device 116 for a user datum 112 record. Computing device 104 authenticates a request for a user datum record 160 utilizing any of the methods as described above including for example authenticating a user identifier such as by checking a hash or authenticating a public/private key pair. Computing device 104 may authenticate a request for a user datum record 160 by authenticating that the remote device 116 that requests a user datum 112 record has permission to request a user datum 112 record. For instance and without limitation, a user may authenticate healthcare professionals such as user's doctor or pharmacist to view user datum 112 records but not user's ex-spouse.

With continued reference to FIG. 5, at step 535 computing device 104 retrieves a digitally signed assertion 128 containing at least an unspecified datum 124. This may be performed utilizing any methodology as described above in reference to FIGS. 1-4.

With continued reference to FIG. 5, at step 540 computing device 104 transmits a digitally signed assertion 128 containing an unspecified datum 124 to remote device 116. Transmission may occur utilizing any network methodology as described herein. Computing device 104 may transmit a cannabis guidance output 152 to a remote device 116. Computing device 104 may receive a cannabis training set 144. Cannabis training set 144 may include any of the training data as described above in reference to FIG. 1. Cannabis training set 144 may include a plurality of first data entries, each first data entry of the plurality of first data entries including a user attribute label and a correlated cannabis article label. Computing device 104 creates a first machine-learning model 148 relating an input that includes a user attribute datum to an output that includes a cannabis article using a cannabis training set 144 and a machine-learning algorithm. First machine-learning model 148 includes any of the machine-learning model 148 as described above in reference to FIG. 1. A user attribute datum may include any of the user attributes as described above, including for example a user history datum, a user medical condition and the like. Computing device 104 generates a cannabis guidance output 152 using cannabis training set 144 and a first machine-learning model 148. Computing device 104 transmits a cannabis guidance output 152 to a remote device 116. Computing device 104 may transmit a cannabis guidance output 152 to a remote device 116 utilizing any network transmission methodology as described herein. Cannabis guidance output 152 may include one or more suggestions about cannabis products that a user may consider consuming. For instance and without limitation, a user attribute datum that contains a user medical condition such as insomnia may be utilizing by computing device 104 in combination with cannabis training set 144 and a first machine-learning algorithm to generate an output that includes a cannabis guidance output 152 that recommends a cannabis guidance output 152 that suggests cannabidiol (CBD) cream applied to temples prior to sleep.

With continued reference to FIG. 5, computing device 104 may receive a request from a remote device 116 for a plurality of cannabis article feature 164. Cannabis article feature 164 may include any of the cannabis article feature 164 as described above in reference to FIG. 1. Cannabis article feature 164 may include any filter that may be applied to any data stored on the temporal sequential listing. Computing device 104 filters a plurality of digitally signed assertions to locate digitally signed assertion 128 that contain a cannabis article feature 164. Computing device 104 retrieves a located digitally signed assertion 128 that contains a cannabis article feature 164 and transmits the located digitally signed assertion 128 wherein the located digitally signed assertion 128 contain an unspecified datum 124.

With continued reference to FIG. 5, computing device 104 receives an insurance coverage request 168 for a cannabis article from a remote device 116. This may include for example an insurance coverage request 168 from a medical insurance company. Computing device 104 authenticates an insurance coverage request 168 such as by authenticating the identify of a remote device 116 and ensuring that a remote device 116 is allowed access to information pertaining to a user. Computing device 104 transmits a user datum 112 to a remote device 116 that may include information necessary to authorize insurance coverage for a cannabis article.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random-access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

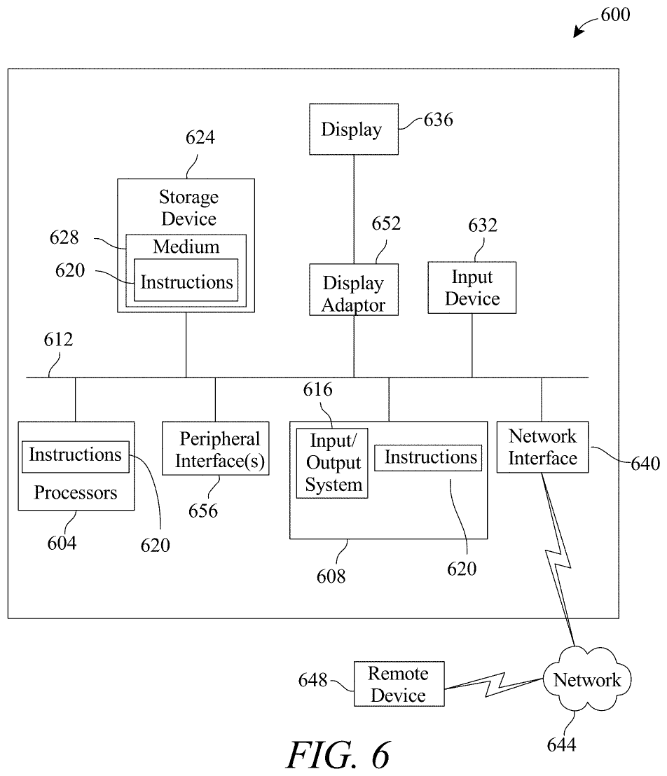

FIG. 6 shows a diagrammatic representation of one embodiment of a computing device in the exemplary form of a computer system 600 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 600 includes a processor 604 and a memory 608 that communicate with each other, and with other components, via a bus 612. Bus 612 may include any of several types of bus structures including, but not limited to, a memory bus, a memory controller, a peripheral bus, a local bus, and any combinations thereof, using any of a variety of bus architectures.

Memory 608 may include various components (e.g., machine-readable media) including, but not limited to, a random-access memory component, a read only component, and any combinations thereof. In one example, a basic input/output system 616 (BIOS), including basic routines that help to transfer information between elements within computer system 600, such as during start-up, may be stored in memory 608. Memory 608 may also include (e.g., stored on one or more machine-readable media) instructions (e.g., software) 620 embodying any one or more of the aspects and/or methodologies of the present disclosure. In another example, memory 608 may further include any number of program modules including, but not limited to, an operating system, one or more application programs, other program modules, program data, and any combinations thereof.

Computer system 600 may also include a storage device 624. Examples of a storage device (e.g., storage device 624) include, but are not limited to, a hard disk drive, a magnetic disk drive, an optical disc drive in combination with an optical medium, a solid-state memory device, and any combinations thereof. Storage device 624 may be connected to bus 612 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 624 (or one or more components thereof) may be removably interfaced with computer system 500 (e.g., via an external port connector (not shown)). Particularly, storage device 624 and an associated machine-readable medium 628 may provide nonvolatile and/or volatile storage of machine-readable instructions, data structures, program modules, and/or other data for computer system 600. In one example, software 620 may reside, completely or partially, within machine-readable medium 628. In another example, software 620 may reside, completely or partially, within processor 604.

Computer system 600 may also include an input device 632. In one example, a user of computer system 600 may enter commands and/or other information into computer system 600 via input device 632. Examples of an input device 632 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 632 may be interfaced to bus 612 via any of a variety of interfaces (not shown) including, but not limited to, a serial interface, a parallel interface, a game port, a USB interface, a FIREWIRE interface, a direct interface to bus 612, and any combinations thereof. Input device 632 may include a touch screen interface that may be a part of or separate from display 636, discussed further below. Input device 632 may be utilized as a user selection device for selecting one or more graphical representations in a graphical interface as described above.

A user may also input commands and/or other information to computer system 600 via storage device 624 (e.g., a removable disk drive, a flash drive, etc.) and/or network interface device 640. A network interface device, such as network interface device 640, may be utilized for connecting computer system 600 to one or more of a variety of networks, such as network 644, and one or more remote device 116 648 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 644, may employ a wired and/or a wireless mode of communication. In general, any network topology may be used. Information (e.g., data, software 620, etc.) may be communicated to and/or from computer system 600 via network interface device 640.

Computer system 600 may further include a video display adapter 652 for communicating a displayable image to a display device, such as display device 636. Examples of a display device include, but are not limited to, a liquid crystal display (LCD), a cathode ray tube (CRT), a plasma display, a light emitting diode (LED) display, and any combinations thereof. Display adapter 652 and display device 636 may be utilized in combination with processor 604 to provide graphical representations of aspects of the present disclosure. In addition to a display device, computer system 600 may include one or more other peripheral output devices including, but not limited to, an audio speaker, a printer, and any combinations thereof. Such peripheral output devices may be connected to bus 612 via a peripheral interface 656. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for tracking the transfer of a cannabis article, the system comprising:
a computing device the computing device further comprising:
a repository module, the repository module designed and configured to:
receive at least a user datum from a remote device, wherein the at least a user datum includes at least a first user cannabis article;
a repository engine operating on the repository module the repository engine designed and configured to:
receive the at least a user datum from the repository module;
create at least an unspecified datum as a function of the at least a user datum;
generate a digitally signed assertion containing the at least an unspecified datum and a cannabis guidance output; and
insert the digitally signed assertion in a temporally sequential listing; and
a transmission module the transmission module designed and configured to:
receive a request from a remote device for a user datum record;
authenticate the request from the remote device;
retrieve the digitally signed assertion containing the at least an unspecified datum and the cannabis guidance output from the repository engine; and
transmit the digitally signed assertion containing the at least an unspecified datum to the remote device.

2. The system of claim 1, wherein the at least a user datum further includes a vigor history detail.

3. The system of claim 1, wherein creating the at least an unspecified datum further comprises:
removing personal identifying data pertaining to the user from the at least a user datum;
generating a digitally signed assertion containing a unique identifier for the user;
inserting the digitally signed assertion containing the unique identifier for the user in the temporary sequential listing; and
storing the user identifying data in the repository module.

4. The system of claim 1, wherein generating the digitally signed assertion further comprises:
creating a secure timestamp when the at least a user datum is received from the remote device; and
generating the digitally signed assertion containing the secure timestamp.

5. The system of claim 1, wherein the repository module is further configured to:
receive at least an expert datum from a remote device wherein the at least an expert datum includes a user identifier;
generate an expert guidance datum;
locate a temporal sequential listing relating to the user as a function of the user identifier;
generate a digitally signed assertion containing the expert guidance datum; and
store the digitally signed assertion containing the expert guidance datum linked to the user.

6. The system of claim 5, wherein generating the expert guidance datum further includes:
mapping, at a language processing module operating on the repository module, the at least an expert datum to a user condition.

7. The system of claim 1, wherein the repository module is further designed and configured to:
receive a cannabis training set including a plurality of first data entries, each first data entry of the plurality of first data entries including at least a user attribute label and at least a correlated cannabis article label;
create at least a first machine-learning model relating an input that includes a user attribute datum to an output that includes a cannabis article using the cannabis training set and a machine-learning algorithm;
generate the cannabis guidance output using the cannabis training set and the first machine-learning model; and
transmit the cannabis guidance output to a remote device.

8. The system of claim 1, wherein the transmission module is further configured to:
receive a request from a remote device for a plurality of cannabis article features;
filter a plurality of digitally signed assertions to locate digitally signed assertions that contain the cannabis article features;
retrieve located digitally signed assertions that contain the cannabis article features; and
transmit the located digitally signed assertions wherein the located digitally signed assertions contain an unspecified datum.

9. The system of claim 1, wherein the transmission module is further configured to:
receive a coverage request for a cannabis article from a remote device;
authenticate the coverage request; and
transmit the at least a user datum to the remote device.

10. The system of claim 1, further comprising a dispensary computing remote device, the dispensary computing remote device configured to:
generate a user datum containing at least a first user cannabis article;
display a cannabis tracking consent agreement;
receive a user response containing a positive cannabis tracking consent agreement;
prompt a user informed advisor identification;
receive an informed advisor identification; and
transmit the user datum containing the at least a first user cannabis article and the informed advisor identification to a computing device.

11. The system of claim 1, further comprising a professional computing remote device, the professional computing remote device configured to:
receive from a computing device a digitally signed assertion containing a first user datum;
generate a second user datum wherein the second user datum contains a response to the first user datum;
link the first user datum and the second user datum; and
transmit the linked first user datum and the second user datum to the computing device.

12. A method of tracking the transfer of a cannabis article, the method comprising:
receiving at a computing device at least a user datum from a remote device, wherein the at least a user datum includes at least a first user cannabis article;
creating by the computing device at least an unspecified datum as a function of the at least a user datum;
generating by the computing device a digitally signed assertion containing the at least an unspecified datum and a cannabis guidance output;
inserting by the computing device the digitally signed assertion in a temporally sequential listing;
receiving by the computing device a request from a remote device for a user datum record;
authenticating by the computing device the request from the remote device;
retrieving by the computing device the digitally signed assertion containing the at least an unspecified datum and the cannabis guidance output; and
transmitting by the computing device the digitally signed assertion containing the at least an unspecified datum to the remote device.

13. The method of claim 12, wherein the at least a user datum further includes a vigor history datum.

14. The method of claim 12, wherein creating the at least an unspecified datum further comprises:
removing personal identifying data pertaining to the user from the at least a user datum;
generating a digitally signed assertion containing a unique identifier for the user;
inserting the digitally signed assertion containing the unique identifier for the user in the temporary sequential listing; and
storing the user identifying data in the repository module.

15. The method of claim 12, wherein generating the digitally signed assertion further comprises:
creating a secure timestamp when the at least a user datum is received from the remote device; and
generating the digitally signed assertion containing the secure timestamp.

16. The method of claim 12, wherein generating at least a digitally signed assertion further comprises:
receiving at least an expert datum from a remote device wherein the at least an expert datum includes a user identifier;
generating an expert guidance datum;
locating a temporal sequential listing relating to the user as a function of the user identifier;
generating a digitally signed assertion containing the expert guidance datum; and
storing the digitally signed assertion containing the expert guidance datum linked to the user.

17. The method of claim 16, wherein generating the expert guidance datum further includes:
mapping, at a language processing module operating on the computing device, the at least an expert datum to a user condition.

18. The method of claim 12 further comprising:
receiving a cannabis training set including a plurality of first data entries, each first data entry of the plurality of first data entries including at least a user attribute label and at least a correlated cannabis article label;
creating at least a first machine-learning model relating an input that includes a user attribute datum to an output that includes a cannabis article using the cannabis training set and a machine-learning algorithm;
generating the cannabis guidance output using the cannabis training set and the first machine-learning model; and
transmitting the cannabis guidance output to a remote device.

19. The method of claim 12 further comprising:
receiving a request from a remote device for a plurality of cannabis article features;
filtering a plurality of digitally signed assertions to locate digitally signed assertions that contain the cannabis article features;
retrieving the located digitally signed assertions that contain the cannabis article features; and
transmitting the located digitally signed assertions wherein the located digitally signed assertions contain an unspecified datum.

20. The method of claim 12 further comprising:
receiving a coverage request for a cannabis article from a remote device;
authenticating the coverage request; and
transmitting the at least a user datum to the remote device.
